(19) United States
(12) Patent Application Publication
(10) Pub. No.: US 2008/0077104 A1
(43) Pub. Date: Mar. 27, 2008

(54) Absorbent article wrapper component having disposal means

(76) Inventors: Noah J. Baer, Appleton, WI (US); Michael J. Bott, Oshkosh, WI (US); Stuart J. Burgan, Appleton, WI (US); James J. Clark, Appleton, WI (US); Susan J. Moder, Appleton, WI (US); Carrie N. Pateras, Boardman, OH (US); Donald J. Sanders, Larsen, WI (US); Jessica L. Ustishen, Kalamazoo, MI (US)

Correspondence Address: KIMBERLY-CLARK WORLDWIDE, INC.;Catherine E. Wolf, 401 NORTH LAKE STREET, NEENAH, WI, 54956, US

(21) Appl. No.: 11/525,410

(22) Filed: Sep. 22, 2006

(51) Int. Cl. A61F 13/15

(52) U.S. Cl. 604/385.13; 604/385.02; 604/385.06

(57) ABSTRACT

The present invention provides users of absorbent personal care articles with a convenient and discreet way to dispose of used absorbent personal care articles. In one disclosed embodiment, a wrapper component for an absorbent personal care article has a disposal device attached to, held adjacent to or contained within an interior volume of the wrapper component in a way that a user of an absorbent personal care article will be able to access the disposal device prior to handling the unused absorbent personal care article. Provided is an easy and discreet means to wrap or conceal used absorbent personal care articles.

### FIELD OF THE INVENTION

The present invention relates generally to a wrapper component for a disposable absorbent article.

### BACKGROUND OF THE INVENTION

Absorbent personal care articles intended to absorb discharged body fluids are well known in the art. Such absorbent articles generally comprise a fibrous mass or other absorbent body which can absorb and hold body fluids. Similarly, it is well known that feminine care articles have been employed to absorb and hold liquids, such as urine and/or menses. The absorbent articles have included various systems of liquid-handling layers, such as intake layers, distribution layers, retention layers and the like. Typically, absorbent articles are placed in purses, backpacks, briefcases, and the like, such that they are loose and are free to move within the purses, backpacks, briefcases, and the like with the other items contained therein. Unfortunately, purses, backpacks, briefcases, and the like do not always provide a hygienic environment for the articles, and thus the articles can become dirty and/or damaged. To avoid the problems described above, often personal care products, including for example, feminine pads, tampons and pantiliners and the like, are individually wrapped in a pouch or similar wrapper, which generally includes a flap that closes the pouch or wrapper.

Currently, wrapper components or pouches for absorbent personal care articles do not provide a user with a convenient and discreet way to dispose of a used absorbent article. When an absorbent personal care article that is currently being worn or used is in need of changing, some users will use the wrapper component or pouch holding the new or unused absorbent article to wrap or conceal the used absorbent personal care articles, after removing the unused absorbent article from the wrapper component or pouch. Using the wrapper presents the problem that the unused absorbent personal care article must be removed prior to placing the used article in the wrapper, which often results in the user handling both the used and unused absorbent personal care articles at the same time, or forcing the user to remove the unused absorbent personal care article and place the unused absorbent personal care article in a location to prevent soiling. On occasion, this action may cause the unused absorbent personal care article to become soiled or contaminated prior to use. In addition, the wrapper used to wrap the unused absorbent article may be damaged during opening, making the wrapper unusable to effectively wrap or conceal the used absorbent article.

Some users of absorbent personal care articles pack a separate bag to seal the used absorbent article for purposes of disposal. Other users of absorbent personal care articles wrap the used absorbent personal care article in tissue paper or facial tissue to conceal the used absorbent article, while some users dispose of the used absorbent personal care article without wrapping the used article. Each of these disposal options has disadvantages. For example, in the case where the user packs a separate bag for disposing of a used absorbent personal care article, the user needs to remember to replace the bag once used or filled. In the case where the user places the used absorbent personal care article in tissue paper and facial tissue, the tissue paper or facial tissue may be effective at concealing the used absorbent article initially; however, over time the tissue paper or facial tissue may become wet with bodily fluid, which may leak from the absorbent article or be absorbed by the tissue paper or facial tissue. When this occurs, the absorbent personal care article will no longer be concealed by the tissue paper or the facial tissue. Disposing of the used absorbent personal care article without wrapping the used absorbent personal care article does not provide the user with discreetness, since the used article may be viewed by others.

Often users of absorbent personal care articles, when changing these articles in pubic restrooms or in the home of another, often desire to take the used absorbent care article with them rather than leave it in the wastebasket often found in bathrooms of individual residences and public restrooms. This is because the use of absorbent personal care articles often has a stigma associated with the articles. The current methods of wrapping absorbent personal care articles described above do not provide an easy way for the user to take the used absorbent personal care article with them after use, for example, placing the used article in a purse, since the used article may have an odor associated with the bodily fluid absorbed by the absorbent personal care article, or the absorbent personal care article may leak the bodily fluid in the purse or other carrying means.

There is a need in the art to provide users with an easy and discreet means to wrap or conceal used absorbent personal care articles, which overcomes the drawbacks of the current methods used by users of absorbent personal care articles.

### SUMMARY OF THE INVENTION

Generally stated, the present invention provides users of absorbent personal care articles with a convenient and discreet way to dispose of used absorbent personal care articles.

In one embodiment of the present invention provided, is a wrapped absorbent personal care article. The absorbent personal care article is placed in a wrapper component. The wrapper component has an exterior surface and an interior volume and the absorbent personal care article is placed within the interior volume. The wrapper component also has a disposal device, configured to dispose of a used absorbent personal care article which the absorbent personal care article is within the interior volume of the wrapper component is intended to replace. This disposal device is attached to, held adjacent to or contained within the interior volume of the wrapper component in a way that a user of an absorbent personal care article will be able to access the disposal device prior to handling the unused absorbent personal care article.

The disposal device of the present invention may be prepared from a wide variety of materials. Examples of the material include, for example, a web of a material selected from a polymeric film, a metal foil, a nonwoven web or a laminate or composite thereof. The disposal device may have a sheet-like structure or may have a bag-like structure. When the disposal device has a bag-like structure, the structure may be provided with a sealing device which allows the used absorbent personal care article

In another embodiment of the present invention, provided is a wrapped absorbent article. The wrapper component has a bottom edge, a top edge, a first side edge, a second side edge, an interior volume, and an opening located at or near the top edge. The opening allows access to the interior volume, and a resealing means is located at or near the opening. This resealing means allows the wrapper component to be resealed after opening. The wrapper component is also provided with a tamper-evident seal located at or near the opening. The tamper-evident seal is present to allow a user of an absorbent personal care article disposed within the interior volume of the wrapper component, to visually or tactilely determine if the wrapper component has been previously opened, which would indicate that the absorbent personal care article within the interior volume of the wrapper component may have been previously used.

In one embodiment of the present invention, the resealing means may be an adhesive material, a zipper type closing device or a cohesive material.

The wrapper component of the present invention also provides users of absorbent personal care articles with a discreet method of disposing of a used personal care article. To use the wrapper component of the present invention, provided is a unused absorbent personal care article in the wrapper component, and a disposal device attached to or held adjacent to the wrapper component. The disposal device is removed from the wrapper component and the used absorbent personal care article is removed from the place of use. Once the disposal device is removed from the wrapper component and the used absorbent personal care articles is removed from the place of use, the used absorbent personal care article is placed onto or into the disposal device to form a used absorbent personal care article disposal device combination. Next, the used absorbent personal care article disposal device combination is placed into a waste receptacle.

By providing the disposal device of the present invention, the used absorbent personal care article and disposal device combination may be placed into a personal carrying device or a pocket in an article of clothing worn by the user, prior to disposing of the used absorbent personal care article-disposal device combination. This gives the users the option to discreetly dispose of absorbent personal care articles.

### BRIEF DESCRIPTION OF THE DRAWINGS

FIG. 1 shows a possible configuration for an absorbent personal care article which may be provided or placed in the wrapper component of the present invention.

FIG. 2 is a perspective view of a product component in a partially folded configuration.

FIG. 3A is a perspective back-side view of an embodiment of an exemplary individual wrapper component with a pouch to retain the disposal device to the wrapper.

FIG. 3B is a side cross-section of an embodiment of an exemplary individual wrapper component with a pouch to retain the disposal device to the wrapper.

FIG. 4 is a perspective front-side view of an embodiment of an exemplary individual wrapper component.

FIG. 5 is a perspective view of an exemplary individual wrapper component in an open configuration with an absorbent product component in a folded configuration and the disposal device is located on the inner side of the flap.

FIG. 6A is a plan view of a second exemplary wrapper component in an open configuration with a personal care product component in an unfolded configuration.

FIG. 6B is a perspective view of the second wrapper component in a partially open configuration with the personal care product component in a partially folded configuration.

FIG. 7A is a perspective view of a disposal device having a sheet-like structure.

FIG. 7B is a perspective view of a disposal device having a bag-like structure.

FIG. 7C shows a side cut-away view of the disposal device of FIG. 7B.

FIG. 8A shows a side cut-away view of the wrapper component of the embodiment of the present invention with a tamper-evident seal.

FIG. 8B shows a side cut-away view of the wrapper component of the embodiment of the present invention with a tamper-evident seal.

FIG. 8C shows a side cut-away view of the wrapper component of the embodiment of the present invention with a tamper-evident seal.

FIG. 8D shows a front-side view of the wrapper component of the embodiment of the present invention with a tamper-evident seal.

### DEFINITIONS

It should be noted that, when employed in the present disclosure, the terms “comprises”, “comprising” and other derivatives from the root term “comprise” are intended to be open-ended terms that specify the presence of any stated features, elements, integers, steps, or components, and are not intended to preclude the presence or addition of one or more other features, elements, integers, steps, components, or groups thereof.

It should be understood that the term “absorbent personal care product” or “absorbent personal care article” as used herein refers to any article used to control bodily fluids, and includes “absorbent products,” which refers to any article configured to absorb and retain bodily exudates, including urine, bowel movements, blood and menses, and includes such a product in a packaged and unpackaged configuration. As such, personal care products, as used herein, includes without limitation, diapers, child toilet training pants, adult incontinence garments, male incontinence products, tampons, vaginal suppositories, pantiliners, pads, sanitary napkins, tissues, wipes, etc. Examples of commercially available personal care products include, without limitation, Poise® feminine care products, including pantiliners and pads, and Kotex® feminine care products, including pads, tampons and liners, all available from Kimberly-Clark Global Sales, Neenah, Wis. Other absorbent personal care articles included, for example, Depend® absorbent personal care articles, such as Depend Guards for Men and Boost™ products, also available from Kimberly-Clark Global Sales, Neenah, Wis.

As used herein, the term “nonwoven fabric or web” means a web having a structure of individual fibers or threads which are interlaid, but not in a regular or identifiable manner as in a knitted fabric. The term also includes individual filaments and strands, yarns or tows as well as foams and films that have been fibrillated, apertured, or otherwise treated to impart fabric-like properties. Nonwoven fabrics or webs have been formed from many processes such as for example, meltblowing processes, spunbonding processes, airlaying processes and bonded carded web processes. The basis weight of nonwoven fabrics is usually expressed in ounces of material per square yard (osy) or grams per square meter (gsm) and the fiber diameters useful are usually expressed in microns. (Note that to convert from osy to gsm, multiply osy by 33.91).

As used herein, the term “spunbonded web” refers to a web of small diameter fibers which are formed by extruding molten thermoplastic material as filaments from a plurality of fine, usually circular capillaries of a spinneret with the diameter of the extruded filaments then being rapidly reduced as, for example, described in U.S. Pat. No. 4,340,563 to Appel et al., and U.S. Pat. No. 3,692,618 to Dorschner et al., U.S. Pat. No. 3,802,817 to Matsuki et al., U.S. Pat. Nos. 3,338,992 and 3,341,394 to Kinney, U.S. Pat. No. 3,502,763 to Hartmann, and U.S. Pat. No. 3,542,615 to Dobo et al. Spunbond fibers are quenched and generally not tacky when they are deposited onto a collecting surface. Spunbond fibers are generally continuous and have average diameters frequently larger than 7 microns, more particularly, between about 10 and 20 microns.

As used herein, the term “meltblown web” means a web of fibers formed by extruding a molten thermoplastic material through a plurality of fine, usually circular, die capillaries as molten threads or filaments into converging high velocity, usually heated, gas (e.g. air) streams which attenuate the filaments of molten thermoplastic material to reduce their diameter, which may be to microfiber diameter. Thereafter, the meltblown fibers are carried by the high velocity gas stream and are deposited on a collecting surface often while still tacky to form a web of randomly dispersed meltblown fibers. Such a process is disclosed, for example, in U.S. Pat. No. 3,849,241 to Butin. Meltblown fibers are microfibers which may be continuous or discontinuous and are generally smaller than 10 microns in average diameter.

As used herein, the term “coform material” or “coform” generally refers to composite materials comprising a stabilized matrix of thermoplastic fibers and a second non-thermoplastic material. As an example, coform materials may be made by a process in which at least one meltblown die head is arranged near a chute through which pulp and/or other absorbent materials are added to the web while it is forming. Suitable absorbents include, but are not limited to, fibrous organic materials such as woody or non-woody pulp such as cotton, rayon, recycled paper, wood pulp fluff, cellulose and/or cellulosic staple fibers, and also include inorganic absorbent materials such as superabsorbent materials and/or treated polymeric staple fibers. Exemplary coform materials are disclosed in commonly assigned U.S. Pat. No. 5,284,703 to Everhart et al., U.S. Pat. No. 5,350,624 to Georger et al., and U.S. Pat. No. 4,100,324 to Anderson et al.; the entire contents of each of the aforesaid references are incorporated herein by reference.

As used herein, “bonded carded webs” or “BCW” refers to nonwoven webs formed by carding processes as are known to those skilled in the art and further described, for example, in coassigned U.S. Pat. No. 4,488,928 to Alikhan et al. which is incorporated herein in its entirety by reference. Briefly, carding processes involve starting with a blend of, for example, staple fibers with bonding fibers or other bonding components in a bulky batt that is combed or otherwise treated to provide a generally uniform basis weight. This web is heated or otherwise treated to activate the adhesive component resulting in an integrated, usually lofty nonwoven material.

“Airlaying” or “airlaid web” is a well known process by which a fibrous nonwoven layer can be formed. In the airlaying process, bundles of small fibers are separated and entrained in an air supply and then deposited onto a forming screen, usually with the assistance of a vacuum supply. The randomly deposited fibers then are bonded to one another using, for example, hot air or a spray adhesive.

As used herein, the term “polymer” generally includes but is not limited to, homopolymers, copolymers, such as for example, block, graft, random and alternating copolymers, terpolymers, etc. and blends and modifications thereof. Furthermore, unless otherwise specifically limited, the term “polymer” shall include all possible geometrical configurations of the material. These configurations include, but are not limited to isotactic, syndiotactic and random symmetries.

As used herein, “body-facing surface” means that surface of the article or component which is intended to be disposed toward or placed adjacent to the body of the wearer during ordinary use, while the “garment-facing surface” is on the opposite side, and is intended to be disposed to face away from the wearer's body during ordinary use. The outward surface may be arranged to face toward or placed adjacent to the wearer's undergarments when the absorbent article is worn.

As used herein, the term “pattern” refers to images or designs that are constituted by a figure (i.e., a lines, a symbol or character and the like). A pattern desirably has an aesthetic image or design that can provide certain benefits when the absorbent article is viewed by users or consumers of the absorbent articles.

As used herein, the term “connected” is intended to mean directly connected and indirectly connected. By directly connected, it is intended that the connected elements are in contact with one another or affixed to one another. By indirectly connected, it is intended that one or more intervening or intermediate elements are between the two elements which are secured or “connected” together. The intervening elements may be affixed.

### DETAILED DESCRIPTION OF THE INVENTION

Personal care articles such as, for example, feminine care and incontinent absorbent products, generally include a liquid pervious topsheet, a substantially liquid impervious backsheet, and an absorbent core positioned and held between the topsheet and the backsheet. The topsheet is generally operatively permeable to the liquids that are intended to be held or stored by the absorbent article, and the backsheet may be substantially impermeable or otherwise operatively impermeable to the liquids intended to be held or stored by the absorbent article. Disposable absorbent articles may also include other optional components or layers, such as liquid wicking layers, liquid distribution layers, barrier layers, and the like, as well as combinations thereof, which may improve the fluid handling and storage properties of the disposable absorbent article. Generally, disposable absorbent articles and the components thereof provide a body-facing surface and a garment-facing surface. The body-facing surface is generally the topsheet and the garment-facing surface is the backsheet. As an alternative, the substantially liquid impervious backsheet may be replaced with a liquid pervious backsheet, when the absorbent personal care product is used in conjunction with another liquid impervious layer or article, such as, for example, liquid impervious pants.

In the present invention, the absorbent personal care articles of the present invention are placed into a wrapper component. The wrapper component has an exterior surface and an interior volume. The absorbent personal care article is placed within the interior volume of the wrapper component, which serves to keep the absorbent article clean prior to use. In one embodiment of the present invention, associated with the wrapper component is a disposal device, which is attached to or held adjacent to the wrapper component. Alternatively, the disposal device may be placed in the interior volume of the wrapper component. The disposal device provides the user with a convenient and discreet way to dispose of a used absorbent personal care article when a user changes a used absorbent personal care article and replaces the used absorbent personal care article with an unused absorbent personal care article. Any absorbent article may be placed in the wrapper component, including diapers, incontinence pants, training pants, and menstrual pants. However, typically the wrapper component of the present invention will be used to hold sanitary napkins, tampons, pantiliners, incontinence pads and the like.

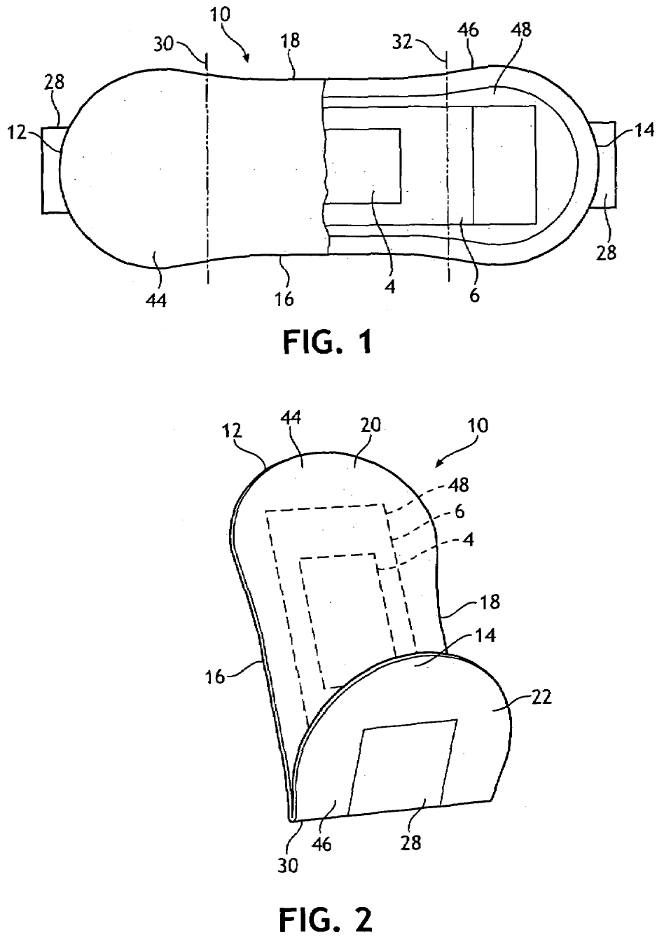

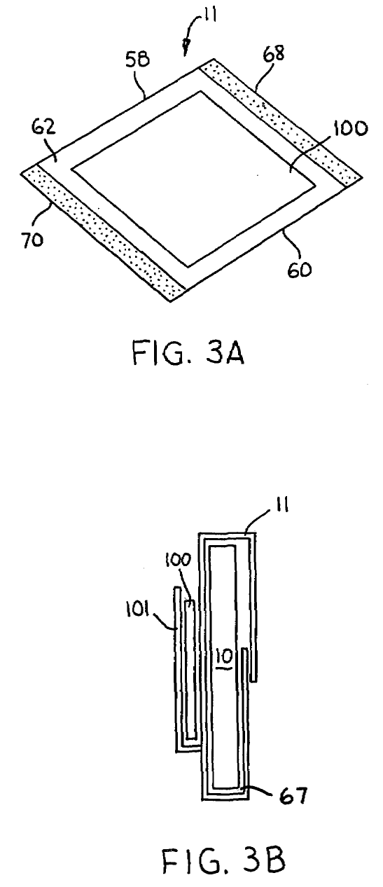

To obtain a better understanding of the absorbent articles which may be present in the present invention, attention is directed to FIGS. 1 and 2. In FIG. 1, an exemplary absorbent product 10 is shown as including an outer cover 46 (otherwise referred to as a baffle or backsheet), an absorbent core 48, an optional tissue layer 6, an optional distribution layer (surge layer) 4 and a body-side liner 44 (also referred to as the topsheet). The absorbent product 10 also has a first side 16 and a second side 18. The first and second sides 16, 18, respectively, are the longitudinal sides of the elongated absorbent product. The sides can be contoured, for example, in a concave shape, or they can be linear. The sides can further include flaps (not shown) that extend laterally outward. Flaps are known in the art and are shown in, for example U.S. Pat. No. 6,387,084 issued to VanGompel et al., which is hereby incorporated by reference in its entirety. In one embodiment (not shown), one or more elastic elements are disposed along the sides to form a gasket with the body of the user. Elastic sides are known in the art, as is shown in U.S. Pat. No. 6,315,765 issued to Datta et al., which is hereby incorporated by reference in its entirety. In one embodiment, the elastic elements are disposed between the body-side liner and the outer cover.

The absorbent product 10 has a first body-side surface 20 and a second garment-side surface 22. Applied to at least a portion of the second garment-side surface 22 is a garment attachment adhesive. In various embodiments, the garment attachment adhesive is configured as a single band of adhesive or as two or more spaced apart strips. Alternatively, the garment attachment adhesive may include a swirl pattern of adhesive which encompasses a major portion of the second garment surface 22 of the absorbent article 10.

A release strip 28, also known as a releasable peel strip, is removably secured to the garment attachment adhesive and serves to prevent premature contamination of the adhesive before the absorbent article 10 is secured to the crotch portion of an undergarment. In various embodiments, the garment attachment adhesive is designed to be secured to the inner crotch portion of an undergarment so as to keep the absorbent product in register with the body of the user. The release strip 28 may extend beyond one or both of the ends 12, 14 of the outer cover, as shown in FIG. 1. Alternatively, the release strip may be as short as the length of the garment attachment adhesive, or slightly longer than the adhesive or may be only as long as the garment attachment adhesive, but does not extend beyond the ends 12 and 14 of the outer cover, as shown in FIG. 2.

The body-side liner or topsheet 44, which is preferably liquid permeable, may be formed from one or more materials. The body-side liner or topsheet 44 must be able to manage different body excretions depending on the type of product. In feminine care products, often the body-side liner or body-contacting layer must be able to handle menses and urine. In the present invention, the body-side liner or topsheet 44 may include a layer constructed of any operative material, and may be a composite material. For example, the body-side liner or body-contacting layer can include a woven fabric, a nonwoven fabric, a polymer film, a film-nonwoven fabric laminate or the like, as well as combinations thereof. Examples of a nonwoven fabric useable in the body-side liner or topsheet 44 include, for example, an airlaid nonwoven web, a spunbond nonwoven web, a meltblown nonwoven web, a bonded-carded-web, a hydroentangled nonwoven web, a spunlace web or the like, as well as combinations thereof. Other examples of suitable materials for constructing the body-side liner or topsheet 44 can include rayon, bonded-carded-webs of polyester, polypropylene, polyethylene, nylon, or other heat-bondable fibers finely perforated film webs, net-like materials, and the like, as well as combinations thereof. These webs can be prepared from polymeric materials such as, for example, polyolefins, such as polypropylene and polyethylene and copolymers thereof, polyesters in general including aliphatic esters such as polylactic acid, nylon or any other heat-bondable materials.

Other examples of suitable materials for the body-side liner or topsheet 44 are composite materials of a polymer and a nonwoven fabric material. The composite materials are typically in the form of integral sheets generally formed by the extrusion of a polymer onto a nonwoven web, such as a spunbond material. In a desired arrangement, the liner or body contacting layer 44 can be configured to be operatively liquid-permeable with regard to the liquids that the article is intended to absorb or otherwise handle. The operative liquid-permeability may, for example, be provided by a plurality of pores, perforations, apertures or other openings, as well as combinations thereof, which are present or formed in the liner or body contacting layer. The apertures or other openings can help increase the rate at which bodily liquids can move through the thickness of the liner or body contacting layer and penetrate into the other components of the article (e.g. into the absorbent core 48). The selected arrangement of liquid-permeability is desirably present at least on an operative portion of the body-side liner or topsheet 44 that is appointed for placement on the body-side of the article. The body-side liner or topsheet 44 can provide comfort and conformability, and can function to direct bodily exudates away from the body and toward the absorbent core 48. The body-side liner or topsheet 44 can be configured to retain little or no liquid in its structure, and can be configured to provide a relatively comfortable and non-irritating surface next to the body tissues of a wearer. In the present invention, the topsheet or body-facing surface of each absorbent article may be embossed, printed or otherwise imparted with a pattern.

The baffle or backsheet 46 may include a layer constructed of any operative material, and may or may not have a selected level of liquid-permeability or liquid-impermeability, as desired. In a particular configuration, the baffle or backsheet 46 may be configured to provide an operatively liquid-impermeable baffle structure. The baffle or backsheet 46 may, for example, include a polymeric film, a woven fabric, a nonwoven fabric or the like, as well as combinations or composites thereof. For example, the baffle may include a polymer film laminated to a woven or nonwoven fabric. In a particular feature, the polymer film can be composed of polyethylene, polypropylene, polyester or the like, as well as combinations thereof. Additionally, the polymer film may be micro-embossed, have a printed design, have a printed message to the consumer, and/or may be at least partially colored. Suitably, the baffle or backsheet 46 can operatively permit a sufficient passage of air and moisture vapor out of the article, particularly out of an absorbent (e.g. storage or absorbent core 48) while blocking the passage of bodily liquids. An example of a suitable baffle material can include a breathable, microporous film, such as those described in, for example U.S. Pat. No. 6,045,900 to McCormack et al.

Bicomponent films or other multi-component films can also be used, as well as woven and/or nonwoven fabrics which have been treated to render them operatively liquid-impermeable. Another suitable baffle material can include a closed cell polyolefin foam. For example, a closed-cell polyethylene foam may be employed.

The liquid permeable body-side liner 44 and the liquid-impermeable baffle 46 may be peripherally sealed together to enclose the absorbent core 48 to form the absorbent article 10. Alternatively, the body-side liner or topsheet 44 can be wrapped around both the absorbent 48 and the baffle or backsheet 46 to form a wrapped pad. The body-side liner 44 and baffle 46, and other components of the absorbent product, can be joined for example with adhesive bonds, sonic bonds, thermal bonds, pinning, stitching or any other attachment techniques known in the art, as well as combinations thereof.

The absorbent core 48 is designed to absorb body exudates, including menstrual fluid, blood, urine, and other body fluids. The absorbent core 48 may contain one or more layers of absorbent material. The layers can contain similar materials or different materials. Suitable materials for the absorbent core 48 include, for example, cellulose, wood pulp fluff, rayon, cotton, and meltblown polymers such as polyester, polypropylene or coform. Coform is a meltblown air-formed combination of meltblown polymers, such as polypropylene, and absorbent staple fibers, such as cellulose. A preferred material is wood pulp fluff, for it is low in cost, relatively easy to form, and has good absorbency.

The absorbent core 48 can also be formed from a composite comprised of a hydrophilic material which may be formed from various natural or synthetic fibers, wood pulp fibers, regenerated cellulose or cotton fibers, or a blend of pulp and other fibers. A desired material is an airlaid material.

In one embodiment, the absorbent core 48 also includes a superabsorbent material, in addition to or in place of the hydrophilic material, which increases the ability of the absorbent core to absorb a large amount of fluid in relation to its own weight. Generally stated, the superabsorbent material can be a water-swellable, generally water-insoluble, hydrogel-forming polymeric absorbent material, which is capable of absorbing at least about 15, suitably about 30, and possibly about 60 times or more its weight in physiological saline (e.g. saline with 0.9 wt % NaCl). The superabsorbent materials can be inserted as particles or in sheet form. The superabsorbent material may be biodegradable or bipolar. The hydrogel-forming polymeric absorbent material may be formed from organic hydrogel-forming polymeric material, which may include natural material such as agar, pectin, and guar gum; modified natural materials such as carboxymethyl cellulose, carboxyethyl cellulose, and hydroxypropyl cellulose; and synthetic hydrogel-forming polymers. Synthetic hydrogel-forming polymers include, for example, alkali metal salts of polyacrylic acid, polyacrylamides, polyvinyl alcohol, ethylene maleic anhydride copolymers, polyvinyl ethers, polyvinyl morpholinone, polymers and copolymers of vinyl sulfonic acid, polyacrylates, polyacrylamides, polyvinyl pyridine, and the like. Other suitable hydrogel-forming polymers include hydrolyzed acrylonitrile grafted starch, acrylic acid grafted starch, and isobutylene maleic anhydride copolymers and mixtures thereof. The hydrogel-forming polymers may be lightly crosslinked to render the material substantially water insoluble. Crosslinking may, for example, be by irradiation or covalent, ionic, Van der Waals, or hydrogen bonding. Hydroxyfunctional polymers have been found to be good superabsorbents for sanitary napkins. Such superabsorbents are commercially available from Dow Chemical, Hoechst-Celanese, and Stockhausen, Incorporated, among others, and are a partially neutralized salt of cross-linked copolymer of polyacrylic acid and polyvinyl alcohol having an absorbency under load value above 25 grams of absorbed liquid per gram of absorbent material (g/g). Other types of superabsorbent materials known to those skilled in the art can also be used.

Additional layers or substrates, including for example, the liquid acquisition and distribution layer 4, also referred to as a surge or transfer layer, and an optional tissue layer 6 are also incorporated into the absorbent product, for example between the body-side liner or topsheet 44 and the absorbent core 48. The distribution layer 4 may be shorter than the absorbent core 48 or have the same length as the absorbent core 48. The distribution layer serves to temporarily hold an insulting fluid to allow the absorbent core sufficient time to absorb the fluid, especially when a superabsorbent material is present. In one embodiment, the absorbent core, transfer layer and other components, such as tissue layers, are free floating (unattached) between the outer-cover and the liner, which are secured along only the peripheral edges thereof. Alternatively, the absorbent core, transfer layer and other components are attached to one or both of the outer-cover and liner and/or to each other.

The absorbent article may be folded along a pair of fold lines 30, 32 to form a tri-fold configuration, prior to insertion into the wrapper component. In other embodiments, the absorbent article can be bi-folded, flat or rolled. In whichever configuration the absorbent article is in, the absorbent article is placed into a wrapper component of the present invention. Alternatively, a plurality (meaning two or more) of article components can be disposed in a single wrapper component and a plurality of wrapper components, with absorbent article contained therein, can be placed in an outer packaging component. The outer packaging component is any packaging which is used to transport, store, or protect the absorbent articles and wrapper components. Examples of outer packaging components include bags, boxes and the like. Outer packaging components are typically used to allow consumers a convenient means to transport a plurality of absorbent articles from the store to their home or other places of use.

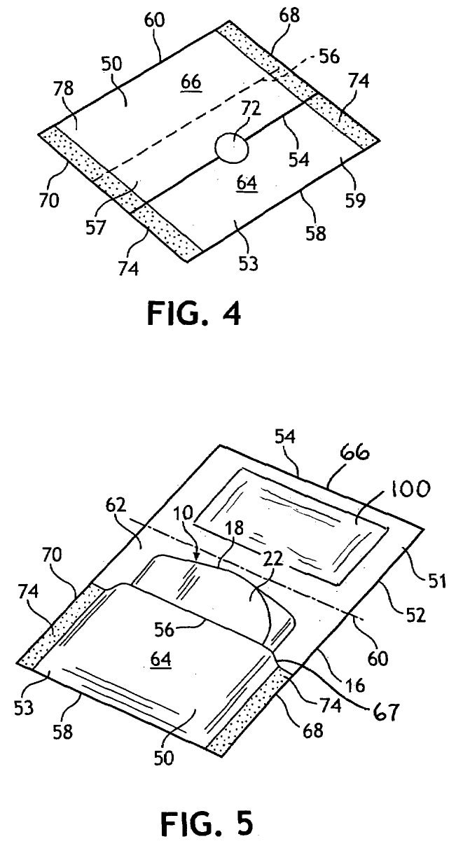

The wrapper component 11 may have a number of different configurations without departing from the scope of the present invention. Although the wrapper component 11 may have different configurations or can be prepared in other ways without departing from the scope of the present invention, referring to FIGS. 3A, 3B, 4 and 5, a typical wrapper component 11 has a pouch 50 formed from a strip or web 52 of material having first and second ends having free edges 56, 54, respectively. It should be understood that the term “free edge” refers to an edge that is unattached after the wrapper component is opened, regardless of whether the free edge is attached when the wrapper component is closed. Each of the first and second ends is folded along fold lines to define the top 60 and bottom edge 58 of the pouch 50, respectively. The folded pouch has a back panel 62, a first panel 64 and a second panel 66. The first panel 64 and back panel 62 may be secured along side edges 68, 70 thereof to form a pocket shape. This pocket shape forms the interior volume 67, which will receive the absorbent article 10. Each wrapper has an interior surface 51 and an exterior surface 53. In one embodiment, the interior volume 67 may be shaped and dimensioned to receive a single product component, which is individually wrapped in the pouch. Alternatively, the interior volume 67 may be configured to hold more than one absorbent article. The second panel 66 is folded over the first panel 64 such that the free edge 54 of the second panel overlies the first panel 64. The first panel 64 has a covered or overlapping portion 57 extending between the free edge 54 (exterior) and the free edge 56 (interior), which covered or overlapped portion 57 underlies the second panel 66. Generally, the second panel 66 may be a flap, but it is not required for the present invention that the second panel 66 be a flap. A flap is a piece of the wrapper component which may be manipulated by a user by grasping the end of the second panel and pulling the second panel upward, towards the top 60.

As an alternative to having the free edge overlap the first panel, the wrapper component can be designed such that there is no overlap between the free edge 54 and the first panel 64, without departing from the scope of the present invention. For example, the free edges 54, 56 may abut each other (not shown) or may be separated by a small distance (also not shown). As such, in this alternative, the second panel is defined merely as another panel. In any event, the wrapper component 11 has an interior volume 67 which is capable of holding the absorbent article 10.

When the second panel overlaps the first panel, in one example, the portion 57 has a length of about 0 mm to about 50 mm, generally about 2-22 mm and typically about 4-10 mm between the free edges 54, 56. In various embodiments, the overlap distance is less than or equal to about 95% of the overall wrapper component 11 length in a closed configuration, more desirably less than or equal to about 35% of the wrapper component length, and more desirably less than or equal to about 20% of the wrapper component length. In various embodiments, the free edge 54 is positioned at a distance from either edge 58, 60 that is greater than or equal to about 10% of the overall length of the wrapper component (in a closed configuration), more desirably greater than or equal to about 30%, and more desirably about 50% of the wrapper component length. The first panel further includes an uncovered second portion 59 extending between the free edge 54 and the bottom edge 58. Of course, it should be understood that the length and width of the article and wrapper components can vary according to the type of article and the size of the article.

A pair of side seals 74 secures the first panel 64 to the back panel 62. The side seals are desirably formed after the first panel is folded over the back panel and the second panel is folded over the back panel and the first panel. Although, it is possible that the first panel could first be sealed to the back panel, and the second panel then sealed to one or both of the back panel and first panel. In an alternative configuration, the second panel is not sealed at the side edges of the first panel and back panel. This embodiment is described in more detail below. The sides may be sealed by any method known to those skilled in the art. Exemplary sealing methods include, for example, adhesive sealing, bonding by the application of heat and pressure, ultrasonic bonding or any other art-known bonding methods. In one embodiment of the present invention, the side seals 74 may be frangible, meaning they can be easily broken such that the second panel 66 can be separated from the first panel 64 and back panel 62, and such that the first panel 64 can be easily separated from the back panel 62, wherein the product component 10 is exposed for removal from the pouch by the user.

In an alternative configuration, one or more free edges may be formed along a perforation line, which is or may be adhered to an underlying layer, with the edge defined by the perforation line being a “free edge” after the perforation line is broken. The free edge can be a single layer cut or formed edge, or can include a double-layer folded edge, or can include an edge formed by a plurality of layers. The wrapper material can be formed from materials, such as, but not limited to, a non-woven material, films, paper, laminates, and/or cloth (including woven) materials, and combinations thereof. For example, the pouch can be made as disclosed in U.S. Pat. No. 6,716,203, to Sorebo et al., the entire disclosure of which is hereby incorporated herein by reference. Suitable laminates useable in the present invention include, for example, spunbond-spunbond laminate (SS), spunbond-meltblown-spunbond laminates (SMS), spunbond-film laminates (SF), and film-film laminates. In one embodiment, the pouch is made of a film/spunbond laminate material available from Kimberly-Clark Corp, and known as HBSTL (“highly breathable stretch thermal laminate”), and which material is further disclosed in U.S. Pat. No. 6,276,032, to Nortman et al., the entire disclosure of which is hereby incorporated herein by reference.

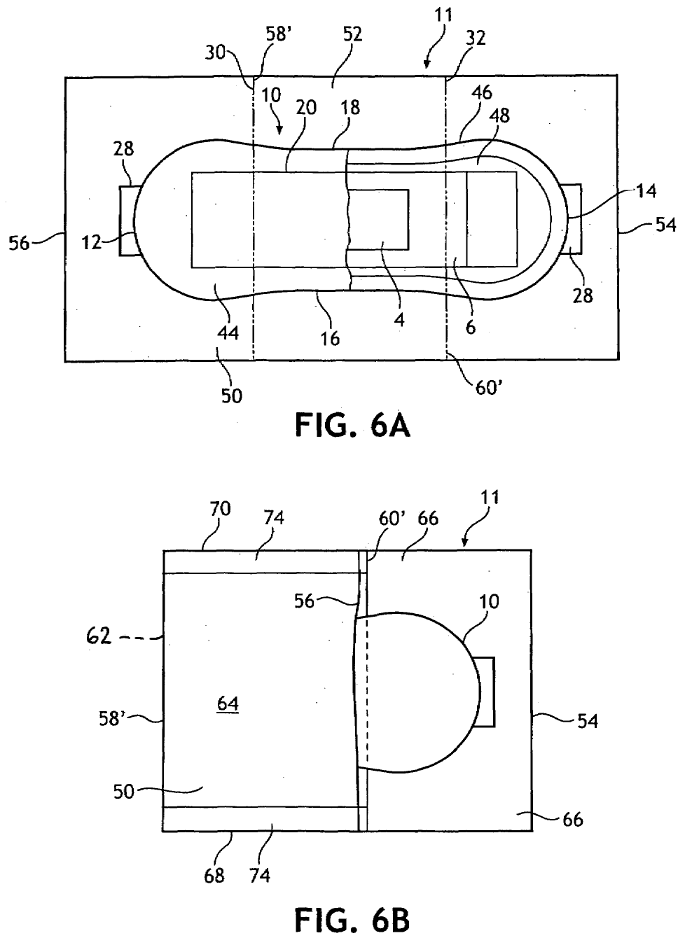

In one alternative embodiment, the second panel 66 is releasably secured to the first panel 64. For example, a fastening element 72, shown as a tab in FIG. 4, is secured across the free edge 54 of the second panel 66 to secure the second panel 66 to the first panel 64. The fastening element can be releasably secured to both of the second panel and first panel, or it can be fixedly secured to one of the second panel and first panel and releasably secured to the other. Other possible configurations include that the fastening element is fixedly secured to both panels and one or both of the panels is provided with an area of weakness, such as a perforated area, which allows a portion of one or both of the panels to be removed or damaged when the wrapper is opened. The fastening element can be formed as adhesive tape, a snap, a button, a mechanical fastener (e.g., hook and loop), a tie, or as any other device known by those skilled in the art. The fastening element can have various alternative shapes, including but not limited to a square, rectangle, triangle, circle, oval, obround, oblong or diamond shape, or any other irregular shape or pattern. In an alternative embodiment, the fastening element is formed on the inside of the second panel such that it engages the first panel as the second panel is folded thereover and is not visible to the user. For example, the fastening of the second panel 66 to the first panel 64 may be accomplished by the use of an adhesive applied to the side of the second panel 66 which contacts the first panel 64, to the side of the first panel 64 which contacts the second panel 66, or both. This adhesive may be applied as a ribbon, dot, a swirl pattern or any other pattern which effectively adheres the second panel 66 to the first panel 64. It is desirable that the adhesive is selected such that the adhesive releasably secures the first and second panels. In another alternative way to fasten the second panel 66 to the first panel 64, the second panel 66 is simply sealed to the first panel 64 with a heat seal or other weld, with the weld defining the fastening element. In another embodiment, the second panel 66 is not sealed or otherwise attached to the first panel 64, but rather is simply folded thereover. Alternatively, the sides of the second panel are sealed to the back panel and to the first panel, with the side seals being breakable in response to a user grasping and lifting the second panel.

In some embodiments, the second panel 66 is refastenably secured to the first panel 64, while in others the second panel 66 is not intended to be secured to the first panel 64 once the wrapper component is opened. For example, in one embodiment, the free edge 54 is defined by a perforation line; with the second panel not being refastenable after the perforation is broken.

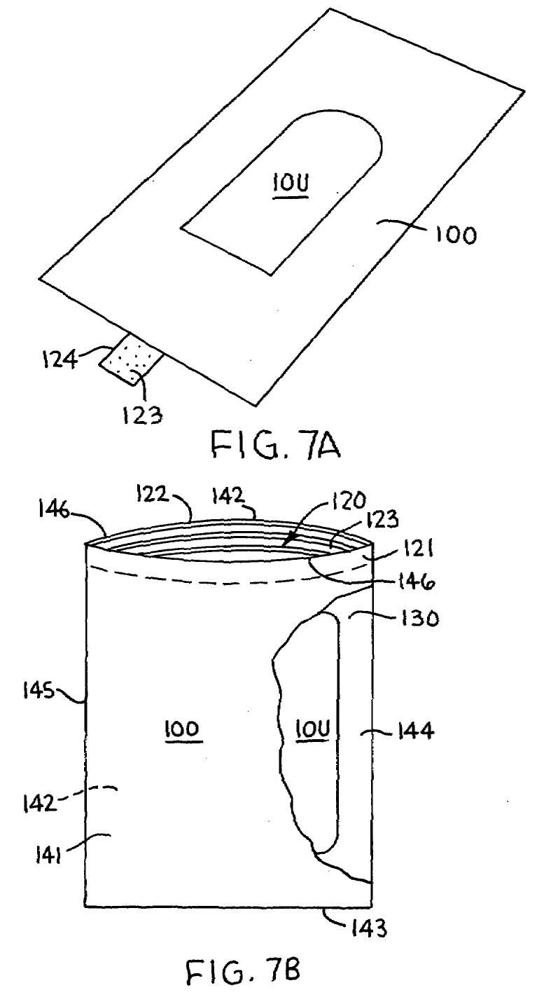

Referring to FIG. 5 the wrapper component is shown. In FIG. 5, the absorbent product 10 is shown in the wrapper component in a folded configuration. For example, the absorbent product can be folded along a pair of fold lines 30, 32, as shown in FIG. 1, to form a tri-fold configuration. In other embodiments, the absorbent product can be bi-folded, flat or rolled. The absorbent product is then inserted into an individual wrapper component, otherwise referred to as a wrapper or pouch. Alternatively, a plurality (meaning two or more) of products 10 can be disposed in a wrapper component. One product/wrapper configuration is shown in U.S. Pat. No. 6,601,706 to McManus et al., which is hereby incorporated by reference. Other wrapper/absorbent product configurations are described in, for example, U.S. Patent Application Publication 2002/0079246 to Ling et al., which is hereby incorporated by reference. The absorbent products can be oriented in various ways within the individual wrapper component, for example, with the fold lines 30, 32 running parallel or perpendicular to the sides of the wrapper component 68, 70.

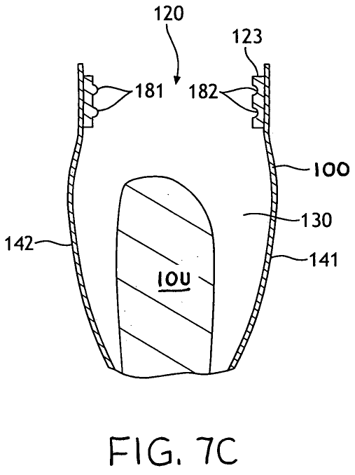

In the present invention, the wrapper component may be preformed or folded with the absorbent article contained in the strip of material used to prepare the wrapper component. As is shown in FIGS. 6A and 6B, the wrapper component 11 is a strip or web 52 of material having first and second ends having free edge 54, 56, respectively. First, the wrapper component and the absorbent article are folded together along line 30 such that the body-side liner or topsheet 44 of the absorbent article comes in contact with itself. In addition, each of sides 68 and 70 come into contact with itself and may be sealed together to form two side seals 74, as is shown in FIG. 6B, or can remain unsealed at this stage. Generally, the wrapper component sides are bonded in the side regions 68 and 70. The sides may be sealed by any method known to those skilled in the art including adhesive sealing, bonding by the application of heat and pressure, ultrasonic bonding or any other art known bonding methods. When folded and sealed as shown in FIG. 6B, the wrapper component has a pouch with a back panel 62, a first panel 64 and a second panel 66. The second panel 66 is then folded over the first panel 64 along line 32 such that the free edge 54 of the second panel overlies the first panel 64. In this configuration, the portion of the topsheet of the absorbent article will be in contact with the first panel 64 of the wrapper component. As with the above described configuration, the first panel 64 has a covered or overlapped portion 57, shown in FIG. 4, extending between the free edge 54 (exterior) and the free edge 56 (interior), wherein the covered or overlapped portion 57 underlies the second panel. It may be advantageous from a materials standpoint to fold the wrapper component and the absorbent article together, since the release strip 28 may be unnecessary since the wrapper component could also be used as the release strip. As with the previously described configurations, the wrapper component may be designed such that there is no overlap between the free edge 54 and the first panel 64, without departing from the scope of the present invention. For example, the free edges 54, 56 may abut each other or may be spread apart by a small distance (not shown). As such, in this alternative, the second panel is defined merely as another panel. In this embodiment of the present invention, the interior volume 67 is formed when the absorbent article 10 and wrapper component 11 are folded together.

In other embodiments, the absorbent article component 10 may be rolled, with a second panel or first panel portion of a wrapper component overlying a panel portion of the wrapper component, regardless of whether the wrapper component is integral with the product component or separate therefrom.

In one embodiment of the present invention, provided is a disposal device which is attached to, held adjacent the wrapper component or is placed within the interior volume of the wrapper component. The disposal device allows a user of absorbent articles to place a used absorbent article within the disposal device and discard the disposal device in a waste basket or similar container. The disposal device could also serve to allow the user to place a used absorbent article in a pocket of apparel worn by the user or to be placed into a purse, a backpack, a brief case or other similar items which may be carried by a user of absorbent personal care articles. The disposal device may be a web of material having a sheet-like structure, in the form of a bag-like structure having an exterior surface, an internal chamber and an opening to allow access to the chamber.

The disposal device may be prepared from a variety of materials. Suitable materials include, for example, polymeric films, metal foils, nonwoven webs or laminates or composites thereof. Examples of polymeric films include polyolefin films such as polyethylene and polypropylene films. Metal films include, for example, tin foil, aluminum foil and the like. Nonwoven webs include the spunbond nonwoven webs, airlaid nonwoven webs, meltblown nonwoven webs and carded nonwoven webs. Suitable laminates and composites useable in the present invention for the disposal device include spunbond-spunbond laminates (SS), spunbond-meltblown-spunbond laminates (SMS), spunbond-film laminates (SF), and film-film laminates. In one embodiment, the pouch is made of a film/spunbond laminate material available from Kimberly-Clark Corp, and known as HBSTL (“highly breathable stretch thermal laminate”), and which material is further disclosed in U.S. Pat. No. 6,276,032, to Nortman et al., the entire disclosure of which is hereby incorporated herein by reference. Generally, a disposal device should have a color or opacity which will effectively disguise or hide the used absorbent personal care article which may be wrapped or contained within the disposal device.

As is shown in FIG. 3A, the disposal device 100 may be directly or indirectly attached to the wrapper component 11. Alternatively, the disposal device 100 may be held adjacent to the wrapper component 11 using a pocket type device 101, shown in FIG. 3B. In addition, the disposal device may be placed within the interior volume 67 of the wrapper component 11. In any event, disposal device 100 should be located in a place on or in the wrapper component so that the user may access the disposal device 100, ideally without handling the unused absorbent personal care article 10 held in the wrapper component. In another embodiment of the present invention, the disposal device 100 may be placed on or adjacent the absorbent personal care article 10 or in the interior volume of the wrapper component 11.

As is shown in FIG. 3 A, the exterior surface 53 of the wrapper component 11 has a disposal device 100 attached thereto. The disposal device may be held to the exterior surface using any mechanism known to those skilled in the art. Suitable mechanisms include, for example, placing an adhesive between the disposable device 100 and the exterior surface 53 of the wrapper component 11. Other mechanisms include using a co-adhesive material to hold the disposal device to the exterior surface 53 of the wrapper component, or a hook and loop type fastener. In addition, the disposal device 100 may be held to an interior surface 51 of the wrapper component 11, using similar methods used to hold the disposal device to the exterior surface of the wrapper component. In one embodiment shown in FIG. 5, the disposal device 100 is held onto the interior surface of the second panel 66 or flap of the wrapper component 11.

As mentioned above, the disposal device may be held adjacent to the wrapper component using a pocket 101, as is shown in FIG. 3B. The pocket 101 may be located on the exterior surface 51 of the wrapper component, as shown in FIG. 3B or may be located on the interior surface 51 of the wrapper component 100 (not shown). The pocket may be prepared from the same material used to prepare the wrapper component or a different material and may be joined to the wrapper component using know processes, such as adhesive bonding, ultrasonic bonding, heat bonding, or the like.

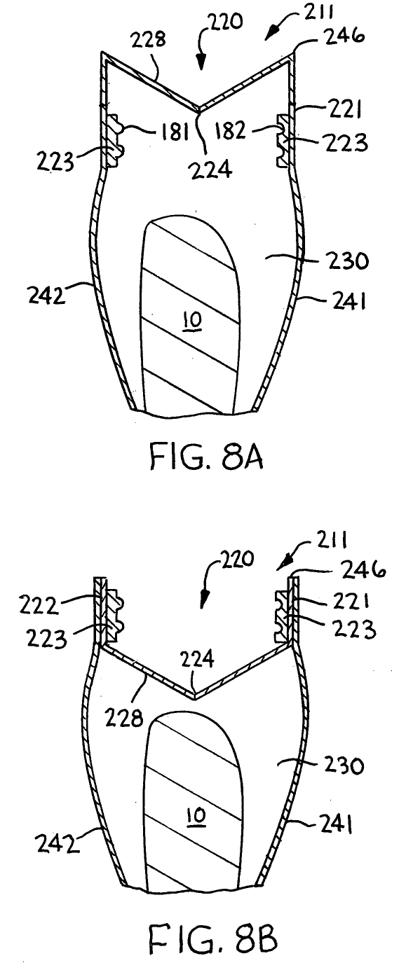

The disposal device 100 may be a sheet of a material described above, as is shown in FIG. 7A, or may be a bag-like structure, shown in FIG. 7B. The bag-like structure, as exemplified in FIG. 7B has an opening 120, when opened, reveals an interior volume 130, but conceals the interior volume 130, when closed. This interior volume 130 is adapted to hold at least one absorbent personal care article 10, and in particular a used absorbent personal care article. The opening 120, has a first side 121 and a second side 122. The interior volume 130 is a defined space or cavity created between the materials used to form the disposal device 100. In the simplest form, the disposal device 100 with an opening 120 has a first panel 141 and a second panel 142. More than two panels may be used to form the disposal device, which could result in a larger chamber being formed. If more than two panels are used, it is generally desired that the opening only has a first side 121 and a second side 122 for ease of opening the opening 120.

The first panel 141 and the second panel 142 each have a bottom edge 143, a first side edge 144, a second side edge 145 and a top edge 146. The bottom edge 143 of the first panel 141 is adjoined to the bottom edge 143 of the second panel 142, the first side edge 144 of the first panel 141 is adjoined to the first side edge 141 of the second panel 142 and the second side edge 144 of the first panel 141 is adjoined to the second side edge 144 of the second panel 142. The opening 120 is located at the top edge 146 of the first panel 141 and the top edge 146 of the second panel 142 such that the first side 121 of the opening is located on the first panel 141 and the second side 122 of the opening is located on the second panel 142. Each panel 141 and 142 may be prepared from separate pieces of material or may be prepared from a unitary piece of material. In the case where the each panel 141 and 142 is made from separate pieces of material, the first and second side edges 143 and 144 of each material and bottom edge must be joined together, either directly or indirectly. An additional piece or pieces of material may be present between each panel 141 and 142 where the panels are joined. Desirably, from the standpoint of cost, the first panel 141 and the second panel 142 are joined directly together. Any method known to those skilled in the art of joining two pieces of material together may be used. Exemplary joining methods include bonding, sealing, stitching and the like. Suitable bonding and sealing methods include, for example, but are not limited to adhesive bonding or sealing, bonding or sealing by the application of heat and pressure, ultrasonic bonding or sealing, or any other art known bonding methods. Alternatively, the material used to prepare the disposal device 100 may be a unitary piece of material. In the case of a unitary piece of material, one of the side edges 144, 145 or the bottom edge 143 does not have to be bonded, sealed or otherwise joined to the corresponding edge on the other panel since the two panels are made from a single piece of material. In the present invention, it is desirable that the material is prepared from a unitary piece of material such that the bottom edges 143 of the panels 141 and 142 do not have to be joined together. In an alternative embodiment, the side edges 144, 145 and bottom edges 143 may be joined directly together or separated by one or more pieces of the same or different types of material. For example, the side edges could be joined together through an expandable material, such as an elastic nonwoven material to expand the side seams of the interior volume 130. Other materials may be used just to increase the capacity of the interior volume 130, or to aid in the bonding or sealing of the panels together.

In the present invention, the disposal device 100 should be of a sufficient size to allow the used absorbent personal care article to be placed in and contained within the device. The actual size would vary, depending on the size or type of absorbent personal care article intended to be disposed of within the disposal device. For example, when the disposal device is a sheet or web of material, as shown in FIG. 7A, generally the size of the disposal device should be such that the length and width is about twice the size of a rolled or folded absorbent personal care article. In the case of a bag-like structure shown in FIG. 7B, the length of the bag or depth of the interior volume should be at least the width of the folded or rolled used absorbent personal care article. The opening 120 should be of a sufficient size such that the used article may be placed in the interior without using much effort by the user. Generally, the size of the opening should be large enough that the used absorbent personal care article can be easily placed into the interior volume of the bag-like structure.

In one embodiment, the disposal device is provided with a closing device which allows the disposal device to be closed by the user. The closing device 123 may be a wide variety of devices known to those skilled in the art, including, for example, adhesive tape 124, shown in FIG. 7A, a snap, a button, a mechanical fastener (e.g., hook and loop), a tie, or any other device known by those skilled in the art. Other closing devices which can be used for closing the opening on a disposal device having a bag-like structure additionally include co-adhesives materials and tongue and groove fasteners. Tongue and groove fasteners are known in the art and are commonly found on disposable zipper closing storage bags. In one embodiment, the disposal device has a closing device and structure that allows for an essentially air tight disposal device, which could prevent odors often associated with used absorbent personal care articles. As is shown in FIG. 7C, the closure device 123 provides a sealing means for the disposal device. Suitable closure devices include tongue 181 and grove 182 sealing mechanisms, and k-style sealing mechanisms. Magnetic sealing mechanisms may also be used. Any mechanism may be used and sealing mechanisms which provide an airtight closure may be desired, from the standpoint of odors.

The disposal device may be attached to or held near the wrapper component in a folded or unfolded condition. Typically, the disposal device will be folded to reduce the size of the disposal device to the size of the wrapper component, in a closed position, or smaller.

To use the disposal device of the present invention, a user removes the disposal device from the wrapper component 11. The user will open or unfold the disposal device such that the used absorbent personal care article may be placed onto or in the disposal device. The user may have removed the used absorbent personal care article from the position of use prior to opening the disposal device or the user may open the disposal device prior to removing the absorbent personal care article. Next the used absorbent personal care article 10U is placed onto the disposal device 100, as is shown in FIG. 7A or is placed into the disposal device 100 as is shown in FIG. 7B and FIG. 7C. In the case where the disposal device is a sheet or web of material, as is shown in FIG. 7A, the sheet or web of material may be rolled or folded around the used absorbent personal care article. If a closing device 123 is provided, the closing device may be used to hold the folded or rolled combination of the disposal device and used absorbent personal care article in a closed position. In the case where the used absorbent personal care article 10U is placed into the disposal device 100, as is shown in FIG. 7B and FIG. 7C, the disposal device may be sealed using the closure device 123. Thereafter, the disposal device is placed into a trash receptacle or placed into a handbag or other similar personal carrying device, or is placed in the pocket of the user, to be disposed of later.

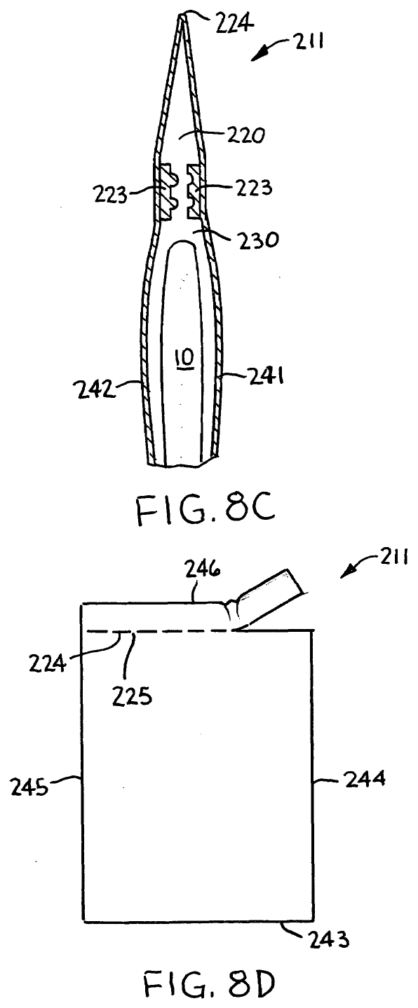

In another embodiment of the present invention, a single wrapper component for both unused and used absorbent articles is provided. In this embodiment of the present invention, the wrapper component stores the new or unused absorbent personal care article and, once the unused absorbent personal care article is removed, the used absorbent personal care article may be placed into the wrapper component. To gain a better understanding of this embodiment of the present invention, attention is directed to FIGS. 8A, 8B, 8C and 8D. In each of these figures, shown is the wrapper component 211. It is noted that the wrapper component of this embodiment of the present invention has a similar structure to the bag-like disposal device shown in FIGS. 7B and 7C.

The wrapper component, as exemplified in FIGS. 8A, 8B, 8C and 8D has an opening 220, when opened, reveals an interior volume 230, but conceals the interior volume 230, when closed. This interior volume 230 is adapted to hold at least one absorbent personal care article 10, and in particular an unused absorbent personal care article prior to opening and a used absorbent personal care article after opening. The opening 220, has a first side 221 and a second side 222. In the simplest form, the wrapper component 211 with an opening 220 has a first panel 241 and a second panel 242. More than two panels may be used to form the wrapper component, which could result in a larger chamber being formed. If more than two panels are used, it is generally desired that the opening only has a first side 221 and a second side 222 for ease of opening the opening 220; however more than two sides may be present at the opening 220.

The first panel 241 and the second panel 242 each have a bottom edge 243, a first side edge 244, a second side edge 245 and a top edge 246. The bottom edge 243 of the first panel 241 is adjoined to the bottom edge 243 of the second panel 242, the first side edge 244 of the first panel 241 is adjoined to the first side edge 244 of the second panel 242 and the second side edge 244 of the first panel 241 is adjoined to the second side edge 244 of the second panel 242. The opening 220 is located at the top edge 246 of the first panel 241 and the top edge 246 of the second panel 242 such that the first side 221 of the opening is located on the first panel 241 and the second side 222 of the opening is located on the second panel 242. Each panel 241 and 242 may be prepared from separate pieces of material or may be prepared from a unitary piece of material. In the case where the each panel 241 and 242 is made from separate pieces of material, the first and second side edges 243 and 244 of each material and bottom edge must be joined together, either directly or indirectly. An additional piece or pieces of material may be present between each panel 241 and 242 where the panels are joined. Additional panels may be used to increase the size of the interior volume 230. Desirably, from the standpoint of cost, the first panel 241 and the second panel 242 are joined directly together. Any method known to those skilled in the art of joining two pieces of material together may be used. Exemplary joining methods include bonding, sealing, stitching and the like. Suitable bonding and sealing methods include, for example, but are not limited to, adhesive bonding or sealing, bonding or sealing by the application of heat and pressure, ultrasonic bonding or sealing, or any other art known bonding methods. Alternatively, the material used to prepare the disposal device 100 may be a unitary piece of material. In the case of a unitary piece of material, one of the side edges 244, 245 or the bottom edge 243 does not have to be bonded, sealed or otherwise joined to the corresponding edge on the other panel since the two panels are made from a single piece of material. In the present invention, it is desirable that the material is prepared from a unitary piece of material such that the bottom edge 243 of the panels 241 and 242 do not have to be joined together. In an alternative embodiment, the side edges 244, 245 and bottom edge 243 may be joined directly together or separated by one or more pieces of the same or different types of material. For example, the side edges could be joined together through an expandable material, such as an elastic nonwoven material to expand the side seams of the interior volume 230. Other materials may be used just to increase the capacity of the interior volume 230, or to aid in the bonding or sealing of the panels together.

The opening 220 of the wrapper component allow access to the interior volume 230 of the wrapper component 211. The opening allows a user to remove a new or unused absorbent personal care article 10 and place a used absorbent article within the interior volume 230 after the new or unused absorbent personal care article has been removed from the interior volume 230. As is mentioned above, typically used absorbent personal care articles have a malodor associated with these used articles. In the present invention, the wrapper component 211 is provided with a resealing means 223 located at or near the opening 220 which allows the wrapper component to be resealed after opening.

The resealing means allows the wrapper component to be closed by the user. The resealing means 223 may be a wide variety of devices known to those skilled in the art. Ideally, the resealing means should be a means which will cause the wrapper component to have an essentially air-tight seal, thereby preventing malodors from escaping from the wrapper component when a used absorbent personal care article is placed in the wrapper component. Suitable resealing means include, for example, a mechanical fastener (e.g., hook and loop), pressure-sensitive adhesives, co-adhesive materials and tongue and groove fasteners. Tongue and groove fasteners are known in the art and are commonly found on zippered storage bags. Tongue and grove fasteners are shown in FIGS. 8A, and 8B and include a tongue 181 and a grove 182 sealing mechanism. Other tongue and groove fasteners include k-style sealing mechanisms. Other types of resealing means which are capable of creating an air-tight seal, not specifically mentioned herein, may also be used.

The wrapper component 211 of this embodiment of the present invention also has a tamper-evident seal 224 located at or near the opening 220. The tamper evident seal 224 provides the user with a visual indication whether or not the wrapper component 211 has been previously opened. In the case where the wrapper component 211 is used to store or transport a used absorbent personal care article, until a suitable disposal container can be located, the tamper-evident seal 224 will provide the user with a visual cue that the wrapper component has been previously opened and that the absorbent article in the wrapper component may contain a used absorbent personal care article.

The tamper-evident seal may be any means which will provide the user with a signal that the wrapper component may have been previously opened. Tamper-evident seals may be a tape-like material placed over the opening, and may be a single piece of a material 228 used to make wrapper component that covers the opening having a opening means, as is shown in FIGS. 8A and 8B. In one embodiment, the tamper-evident seal is a piece of material covering the opening 220 of the wrapper component 211 having a series of perforations at some point along the piece of material. As is shown in FIG. 8C and FIG. 8D, the tamper-evident seal 224 is a piece or pieces of the wrapper with a series of perforations 225 to allow a user to break the perforations to access the interior volume of the wrapper component. The perforation could be formed in a manner which will cause the piece of the wrapper component to be removed during opening or the perforations could be formed in a way which would allow the material of the tamper-evident seal 224 to remain attached to the wrapper component

The tamper-evident seal may be located in the opening of the wrapper component between the interior volume and the resealing means 223, as shown in FIG. 8B. Alternatively, the resealing means 223 may be located in the opening of the wrapper component between the tamper-evident seal 224 and the interior volume 230. By having the tamper evident seal above the resealing means, a user can easily look at the wrapper component to see if the wrapper component had been previously opened, without the need of opening the resealing means.

In a further embodiment of the present invention, the wrapper or the disposal device may be imparted with a low friction coating on the internal surfaces of the wrapper or disposal device. The low friction coating may will aid the user in removing an unused absorbent personal care article form the wrapper, when one is present, and placing a used absorbent personal care within the wrapper or disposal device for discreet disposal. Examples of suitable coatings include silicone coatings and the like.

Although the present invention has been described with reference to various embodiments, those skilled in the art will recognize that changes may be made in form and detail without departing from the spirit and scope of the invention. As such, it is intended that the foregoing detailed description be regarded as illustrative rather than limiting and that it is the appended claims, including all equivalents thereof, which are intended to define the scope of the invention.

1. A wrapped absorbent personal care article comprising:
a wrapper component having an exterior surface and interior volume;
an absorbent personal care article disposed within the interior volume of the wrapper component, and
a disposal device attached to, held adjacent to or contained within the interior volume of the wrapper component.

2. The wrapped absorbent personal care article according to claim 1, wherein the disposal device comprises a web of a material selected from a polymeric film, a metal foil, a nonwoven web or a laminate or composite thereof.

3. The wrapped absorbent personal care article according to claim 1. wherein the disposal device is held adjacent to the wrapper component

4. The wrapped absorbent personal care article according to claim 3, wherein the disposal device held adjacent to the wrapper component by a pocket located on the exterior surface of the wrapper component, wherein the disposal device is located in the pocket.

5. The wrapped absorbent personal care article according to claim 1, wherein the disposal device is attached to the wrapper component by an attachment means.

6. The wrapped absorbent personal care article according to claim 5, wherein the attachment means comprises an adhesive.

7. The wrapped absorbent personal care article according to claim I, wherein the disposal device comprises a bag-like structure comprising an opening, an interior volume and an exterior surface.

8. The wrapped absorbent personal care article according to 7, wherein the bag-like structure comprises a web of a material selected from a polymeric film, a metal foil, a nonwoven web or a laminate or composite thereof.

9. The wrapped absorbent personal care article according to 1, wherein disposal device is folded or rolled prior to use.

10. The wrapped absorbent personal care article according to claim 1, wherein the disposal device further comprises a sealing means.

11. The wrapped absorbent personal care article according to claim 7, wherein the bag-like structure further comprises a sealing means.

12. The wrapped absorbent personal care article according to claim 11, wherein the sealing means comprises an adhesive, a tongue and grove locking mechanism, a tie or a combination thereof the sealing means being located at or near the opening of the bag-like structure.

13. A wrapped absorbent article comprising
a wrapper component comprising a bottom edge, a top edge, a first side edge, a second side edge, an interior volume, an opening located at or near the top edge, the opening allows access to the interior volume, a resealing means located at or near the opening, the resealing means allows the wrapper component to be sealed after opening; and a tamper-evident seal located at or near the opening, and
an absorbent personal care article disposed within the interior volume of the wrapper component.

14. The wrapped absorbent personal care article according to claim 13, wherein the tamper-evident seal comprises a series of perforations.

15. The wrapped absorbent personal care article according to claim 13, wherein the wrapper component comprises a web of a material selected from a polymeric film, a metal foil, a nonwoven web or a laminate or composite thereof.

16. The wrapped absorbent personal care article according to claim 13, wherein the resealing means comprises a tongue and groove sealing mechanism.

17. The wrapped absorbent personal care article according to claim 13, wherein the resealing means comprises a co-adhesive.

18. The wrapped absorbent personal care article according to claim 13, wherein the tamper-evident seal is located in the opening of the wrapper component between the interior volume and the resealing means.

19. The wrapped absorbent personal care article according to claim 13, wherein the resealing means is located in the opening of the wrapper components between the tamper-evident seal and the interior volume.

20. The wrapped absorbent personal care article according to claim 19, wherein the resealing means comprises a tongue and groove sealing mechanism or a co-adhesive.

21. A method of disposing of a used personal care article, comprising
providing a wrapped unused absorbent personal care article in wrapper component, the wrapper component having a disposal device attached to or held adjacent to the wrapper component;
removing the disposal device from the wrapper component;
removing a used absorbent personal care article from the place of use;
placing the used absorbent personal care article onto or into the disposal device to form a used absorbent personal care article disposal device combination; and
disposing of the used absorbent personal care article disposal device combination into a waste receptacle.

22. The method of claim 213 further comprising placing the disposal device combination into a personal carrying device or a pocket in an article of clothing worn by the user, prior to disposing of the used absorbent personal care article disposal device combination.

23. The wrapped absorbent personal care product according to claim 1, further comprising an outer packaging component, wherein the wrapper component, absorbent personal care article and disposal device are placed within the outer packaging component.

24. The wrapped absorbent personal care product according to claim 1, further comprising an outer packaging component, wherein the wrapper component and absorbent personal care article are placed within the outer packaging component.
